(19) United States
(12) Patent Application Publication
(10) Pub. No.: US 2009/0088659 A1
(43) Pub. Date: Apr. 2, 2009

(54) Biological Sensing With Haptic Feedback

(75) Inventors: Michael D. Graham, San Jose, CA (US); Christopher J. Ullrich, Santa Cruz, CA (US)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(73) Assignee: Immersion Corporation, San Jose, CA (US)

(21) Appl. No.: 11/862,639

(22) Filed: Sep. 27, 2007

(51) Int. Cl. A61B 5/04

(52) U.S. Cl. 600/545

(57) ABSTRACT

Systems and methods as described the present disclosure are capable of generating haptic feedback in response to sensing a biological signal from a vertebrate. A method according to one embodiment, among others, includes detecting a neural signal from a vertebrate. The method also includes generating a haptic effect on the vertebrate corresponding to the neural signal.

### TECHNICAL FIELD

The present disclosure generally relates to systems and methods for sensing neural activity and other biological information from a living organism.

### BACKGROUND

In recent years, advancements have been made to develop devices that allow a person to control an electronic device using brainwaves or by some other biological activity. For example, these devices may include some type of sensor that can receive biological signals. These signals are used as control inputs into the electronic device to control the device in a variety of ways depending on the type of biological signal detected. Biological signals, as defined in the present application, may refer to neural signals that are received from the brain, spinal cord, or other parts of the nervous system. Biological signals described herein can also refer to activity of the brain, such as changes in blood flow through the brain, or even, in some implementations, other biological activity such as eye movement, facial muscle activity, etc. By receiving biological inputs from a human, an electronic device can react to the input information without the use of other conventional control devices, such as keypads, buttons, keyboards, and computer mice.

Brain-controlled devices can be used by people who may otherwise be incapable of controlling a device by conventional means. For example, a physically disabled person, such as an amputee or quadriplegic, may have the mental ability to work with an electronic device, but may, however, be incapable of controlling an electronic device using physically manipulated input devices because of their physical disability. By simply using the mind, a person can control a special typewriter via an interaction referred to as a Brain-Computer Interface (“BCI”). Also, in the field of computer games, using brain-controlled programs can provide a new level of interaction between the user and the computer.

Although some developments have been made in this field, still more can be made to further advance this technology. Further advancements can allow a person to have greater control of an electronic device using a BCI as opposed to simply using conventional control systems involving physically manipulated input devices.

### SUMMARY

The present disclosure describes systems and methods for actuating a haptic effect upon a living organism related to sensed biological activity of the organism. In one of many embodiments, for example, a method described herein comprises detecting a neural signal from a vertebrate. The method also includes generating a haptic effect on the vertebrate corresponding to the neural signal.

Other various features, advantages, and implementations of the present disclosure, not expressly disclosed herein, will be apparent to one of ordinary skill in the art upon examination of the following detailed description and accompanying drawings. It is intended that such implied variations also be included herein.

### BRIEF DESCRIPTION OF THE DRAWINGS

The components in the following figures are not necessarily drawn to scale. Instead, emphasis is placed upon clearly illustrating the general principles of the present disclosure. Reference characters designating corresponding components are repeated as necessary throughout the figures for the sake of consistency and clarity.

FIG. 1 is a diagram illustrating an input verification system according to an embodiment of the present disclosure.

FIG. 2 is a block diagram of the feedback device shown in FIG. 1 according to one embodiment.

FIG. 3 is a block diagram of the processing device shown in FIG. 2 according to one embodiment.

FIG. 4 is a flow chart illustrating an embodiment of a process for providing haptic feedback to a human in response to detected neural signals from the human.

### DETAILED DESCRIPTION

The development of Brain-Computer Interfaces (“BCIs”) has opened up vast opportunities for improving how a user controls input into a computer. Brainwaves of a user can be sensed and then converted to electrical signals that can be used in an electronic device, such as, for example, a computer, electric wheelchair, etc. In this respect, computer programs or other electronic devices can be controlled by a user simply thinking in a particular way. By focusing or concentrating the brain in a certain manner, a child with attention deficit issues can be trained to stay focused on a particular task. An athlete can learn mental relaxation skills that can help achieve better athletic performance. Medical improvements, as well as entertainment benefits, can also be achieved by the use of BCIs. In addition to BCIs that detect brainwaves, devices can receive other biological input from a user. Although the present disclosure describes inputs being received in the form of “neural signals”, it is intended that other types of biological signals can also be sensed as well, such as eye movement, facial muscle movement, body or muscle movement, muscle contraction, etc.

Although BCIs provide an advanced way to control devices, the only feedback that the user receives to verify that he or she is in control is the actual response of the device itself. This feedback can only be detected by seeing or hearing the actions of the controlled electronic device. For example, by controlling an electric wheelchair using the mind, only the reaction of the wheelchair itself verifies to the user that the user is actually in control. Feedback may be in the form of visual or audible signal from a computer program, such as when a certain event occurs in the program and is visually displayed on a computer monitor. Also, a computer program may include sounds, which can be perceived as feedback. With a BCI, feedback to the user can be important to communicate to the user that the user is controlling the device as intended. In this disclosure, however, systems and methods for providing tactile, vibrotactile, kinesthetic, or “haptic” feedback in response to a neural input are described. The haptic feedback can be in addition to the existing visual or audible feedback. Such haptic feedback is provided to produce a haptic effect upon the user to verify or confirm that the user is indeed controlling the electronic device.

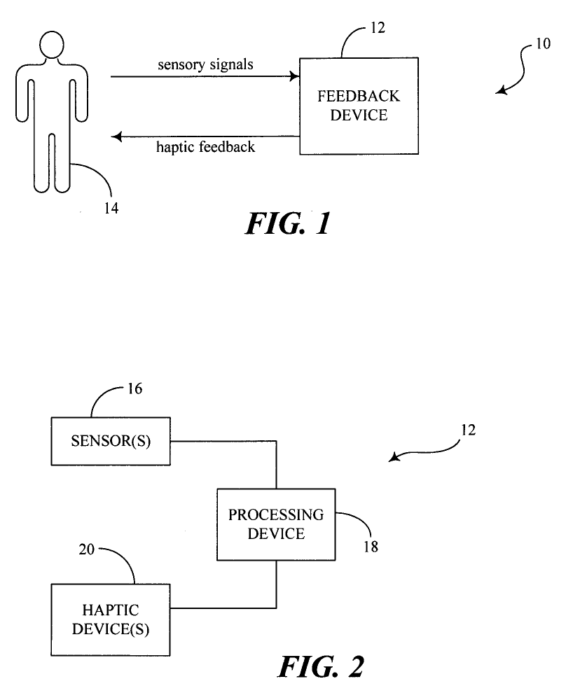

FIG. 1 is a block diagram of an embodiment of an input verification system 10. Input verification system 10 includes a feedback device 12, which receives sensory signals from a human 14 and also provides haptic feedback to human 14. In this disclosure, not only can feedback device 12 interact with humans, as shown in FIG. 1, but it also can interact with any type of mammal or vertebrate. The sensory signals described herein may include neural signals, such as those received from the central nervous system or brain, or may include other signals received from other types of detectable biological activity. For example, other types of biological activity may include eye movement, facial muscle movement, body movement, and/or muscle movement or contraction. In some embodiments, other types of biological activity may include breathing rate, heart rate, and/or other detectable physical characteristics or conditions of human 14.

Feedback device 12 can operate in conjunction with various external electronic devices. Feedback device 12 can be used to detect the sensory signals as described above and then provide electrical signals that are related to the sensed signals to the respective electronic device. In other embodiments, the external electronic device itself may interpret the sensory signals and provide signals to feedback device 12. In still other embodiments, both the electronic device and feedback device 12 may detect and process the sensory signals separately. The detecting and processing of the sensory signals may depend on the particular electronic device with which feedback device 12 operates.

The external electronic device can be controlled by the sensory signals or those signals converted from sensory signals to electrical signals. Feedback device 12 can provide haptic feedback to human 14 in direct response to the detection of the sensory signals. Consequently, human 14 receives a verification or confirmation that the sensory signals have been received.

In other embodiments, feedback device 12 interprets how the control signals affect the external electronic device. Feedback device 12 can detect when an event occurs in the realm of the electronic device that may have some significance with respect to the type of electronic device that is being controlled. For example, if the electronic device is a computer running a computer game and the game includes an explosion, for example, in the realm of the virtual environment, feedback device 12 can detect the virtual explosion and provide haptic feedback stimuli to human 14 so that human 14 can experience the sensation of the explosion, albeit much less severe than an actual explosion.

Input verification system 10 can include numerous applications in numerous fields. In the medical field, sensory signals from human 14 can be used to control a prosthetic device. The prosthetic device can be controlled via feedback device 12 or by another control device customized to interpret the sensory signals with respect to the prosthetic device. Feedback device 12 also receives the sensory signals and provides haptic feedback to a portion of the body of human 14 that is able to feel the haptic stimulus. In this way, human 14 can receive verification that the prosthetic device is controlled as intended, or can even receive a haptic effect indicating possible damage or danger to the prosthetic device or human.

For users with other physical disabilities, feedback device 12 may be used in a system that allows the user to mentally control computer programs or applications without physically manipulated input devices. In some embodiments, a physically disabled user can draw a geometric pattern with the mind, which can be received by feedback device 12. Feedback device 12 can provide a confirmation in the form of haptic feedback when the pattern is complete. In other applications, a blind person can receive haptic feedback by a Braille device that creates a Braille pattern that can be felt.

In the environment of computer games, sounds that result from events in the game can be enhanced with tactile stimulus to human 14. For example, a computer game may include events such as explosions, centrifugal forces upon an avatar in the game, gravitational forces, pain, etc. When these events occur in the realm of the computer game, feedback device 12 can also provide haptic feedback to the player or human 14 to mimic sensations that may be experienced if the events occurred in reality. Furthermore, the feedback provided to the user could be modulated depending on sensory signals from the user. For example, if the feedback device 12 detects that the human 14 is scared in response to an event in the game, the feedback device 12 could apply a first set of haptic effects to the human 14. Otherwise, if the human 14 was not scared, then a different set of haptic effects could be applied.

In sports training, an athlete often works to improve not only physical abilities but also mental toughness. Input verification system 10 can be used in such an environment to receive neural and/or biological signals from the athlete. Based on an interpretation of these signals, feedback device 12 can provide a positive reinforcement in the form of haptic feedback to human 14 when a particular mental state is achieved or when muscles are moving with correct form. Also, negative reinforcement can be provided when the mental state of the athlete is distracted or unfocused or when the body or muscles are not moving in a proper form. Mental training for sports can be an important endeavor to enable an athlete to practice concentration, focus, calmness, relaxation, etc.

Feedback device 12 can also be used in cooperation with a driver alertness device. In this respect, feedback device 12 can sense drowsiness or lack of focus, based on neural signals or even eye movement. In response to a detection of improper alertness of the driver, feedback device 12 can provide haptic feedback to the driver to restore the driver to a safer alertness level. These and other applications of input verification system 10 of FIG. 1 can be envisioned or conceived by one of ordinary skill in the art having read and understood the present disclosure.

FIG. 2 is a block diagram of an embodiment of feedback device 12 shown in FIG. 1. In this embodiment, feedback device 12 includes one or more sensors 16, a processing device 18, and one or more haptic devices 20. Sensors 16 detect or monitor neural activity or other biological activity and convert this sensed activity into electrical signals. Also, sensors 16 may include an amplifying device to amplify the electrical signals if necessary. Sensors 16 then transmit the electrical signals to processing device 18. The communication of signals from sensors 16 to processing device 18 may include wired and/or wireless transmissions. Processing device 18 processes the electrical signals related to the sensed neural or biological activity of human 14. As a confirmation of receipt of the signals, processing device 18 may, in some embodiments, provide signals to haptic devices 20 to indicate to human 14 that the input has been received. In this way, human 14 is affirmed, through haptic feedback, that his or her neural activity has been detected. Also, processing device 18 may provide signals to haptic devices 20 in response to particular events that occur in the realm of an external device being controlled by the neural activity of human 14.

Sensors 16 can include plates or pads that are positioned against or near the skin of the body of human 14. Sensors 16 can also be placed, in some situations, under the skin. When placed under the skin, such as in a medical use, sensors 16 can be permanently or removably attached to portions of the body. For example, sensors 16 can be attached to the brain or other parts of the nervous system to be able to sense neural activity from close range.

In some implementations, sensors 16 can include microwave emitters used for detecting fine movement of eyes, facial muscles, etc. Also, sensors 16 can include an infrared device for sensing changes in blood flow, such as blood flow in the brain. Sensors 16 can include any suitable type of detection device for detecting neural signals and/or a secondary set of inputs, including, for example, eye movement, facial muscle movement, muscle contraction, etc. Sensors 16 can also sense the body's response to a previous haptic effect.

Processing device 18 controls the operations of feedback device 12 by sensing the signals received by sensors 16, processing the sensed signals, and applying haptic response signals to haptic devices 20. In some embodiments, processing device 18 can adjust the haptic response signals that are sent to the actuators depending on the body's response to an earlier haptic effect. In this way, the human completes a loop of the feedback system allowing processing device 18 to adjust the haptic signals to achieve or optimize a desired haptic effect. Processing device 18 can provide feedback when sensory signals are received thereby to communicate to human 14 that the sensory signals have indeed been received. Processing device 18 can also provide feedback when an event occurs within the realm of an electronic device being controlled by human 14. Human 14 therefore receives confirmation when something is in his or her control, when the control has accomplished what it was intended to do, etc.

Processing device 18 may use algorithms to calculate what haptic effects are to be imposed on human 14 and the order in which they are imposed. The algorithms can also calculate the degree, magnitude, frequency, and/or duration of various haptic effects to be imposed, depending on the type or severity of the message or signal to be communicated to human 14.

Haptic devices 20 can be positioned on or near any part of the body of human 14 for providing haptic effects on various parts of the body. The location of a haptic effect on the body, for instance, may relate to predetermined communication patterns between the external device and human 14. Haptic devices 20 may be positioned on the interior of a helmet for contact with the back and top parts of head. Alternatively, haptic devices 20 can be positioned on a band that is wrapped or placed around the back of head and forehead. Haptic devices 20 can be placed or attached to any suitable support structure that can hold haptic devices 20 in a specific orientation with respect to a portion of the body of human 14. Haptic devices 20 can also be positioned on the shoulders, arms, hands, legs, feet, or other parts of the body according to the context of the feedback.

In some embodiments, haptic devices 20 are close enough to the specific nerves of the body to be able to invoke the desired haptic effect. In some embodiments, such as in some medical applications, haptic devices 20 can be embedded under the skin of the human and even placed in direct contact with the nervous system of the human. In this regard, such actuators can be permanently implanted or attached to the human body. Haptic devices 20, in some implementations, can be attached to the same support structure that supports sensors 16. Also, some sensors 16 and haptic devices 20 may share the same space for sensing and actuating the same part of the body.

Haptic devices 20 may include actuators for providing a haptic effect in the form of vibrations. In this respect, the actuators can include any suitable force applying mechanism, electromagnetic mechanism, and/or electromechanical mechanism. For example, an actuator may include an eccentric rotating mass (“ERM”) in which an eccentric mass is rotated by a motor or a linear resonant actuator (“LRA”) in which a mass attached to a spring is driven back and forth. In other embodiments, an actuator may include a piezoelectric circuit, electro-active polymer circuit, shape memory alloy circuit, etc., or other suitable smart material device.

In some embodiments, haptic signals can be applied as electrical current, voltage, or electromagnetic field to portions of the body for invoking a haptic effect on human 14. Normally, haptic devices 20 are configured to provide vibration, pressure, forced muscle contraction, or stimulation of the nervous system or brain to impose the sensation of certain tactile senses. Forced muscle contraction can induce a muscle response that mimics a muscle's response in reality (when used in the virtual realm). In other implementations, haptic devices 20 may be configured to provide additional haptic feedback, such as heat, cold, pain, or other skin sensory stimulus.

Haptic devices 20 can be formed as plates or pads that can be worn or positioned against or in proximity to the skin of the human. Also, haptic devices 20 can include one or more device drivers incorporated therein or alternatively positioned between the processing device 18 and haptic devices 20. In some embodiments, the device drivers can be included in the processing device 18 for providing electrical power to the haptic devices 20.

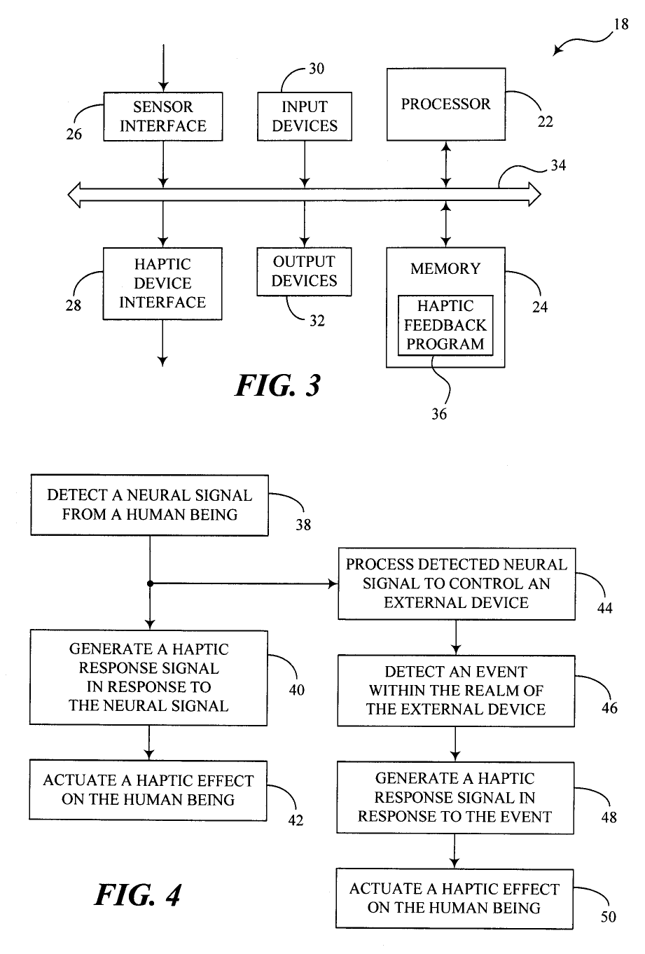

FIG. 3 is a block diagram of an embodiment of the processing device 18 shown in FIG. 2. Processing device 18 in this embodiment includes a processor 22, memory 24, sensor interface 26, haptic device interface 28, input devices 30, and output devices 32. These components of the processing device 18 are interconnected via an internal bus 34 allowing communication therebetween. Memory 24 includes, among other things, a haptic feedback program 36. Haptic feedback program 36 includes instructions for execution by processor 22 to provide haptic feedback to human 14 in response to sensed neural activity.

Sensor interface 26 communicates between the one or more sensors 16 (FIG. 2) and processor 22. From sensors 16, sensor interface 26 receives signals representing the subject's neural activity or other biological input. Input devices 30 may include any suitable mechanism for receiving information. For example, input devices 30 may include computer keyboards, computer mice, or other conventional physically manipulated input devices. These input devices 30 may be used to enter information for selecting a software program to run or for entering preferences, etc. In the medical field, input device 30 may receive input entered by a doctor or medical staff person.

Processor 22 is configured to control the operations of processing device 18 and execute software programs stored in memory 24. For example, by executing haptic feedback program 36, processor 22 is able to provide feedback to human 14 in response to receiving neural activity from human 14. Also, processor 22 can provide feedback in response to an event that occurs in the realm of an electronic device being controlled by the neural activity of the human.

Haptic device interface 28 may include a haptic actuating interface for invoking a haptic effect on human 14. When processor 22 detects a situation when a haptic signal is to be provided to the human, haptic device interface 28 communicates with the one or more haptic devices 20 to impose the appropriate haptic effect upon the human. Depending on the type of haptic device 20 in communication with haptic device interface 28 and the location of haptic device 20 with respect to the body of the human, haptic device interface 28 provides an appropriate signal or signals to induce the intended stimulus.

Output devices 32 may include, for example, a computer monitor for displaying in visual form a graphical image, which may relate to a computer application running on processing device 18. For example, in a game environment, a graphic display may show one or more objects on a screen that are controlled by the user. The events that occur to these objects in the realm of the computer application may be choreographed or synchronized with the haptic effects that are imposed on the human. In this respect, processing device 18 can be a computer or can be associated with a computer, where the computer may include certain peripheral devices as desired. In embodiments where processing device 18 operates in cooperation with a computer as an external electronic device, input devices 30, output devices, and/or bus 34 may be in communication with the external computer. In this respect, the external computer and processing device 18 can share data as needed to enable processing device 18 to apply haptic effects. In some embodiments, processing device 18 may be integrated with a computer such that memory 24 stores not only the haptic feedback program 36 but also stores a computer application to be controlled by human 14. In the computer applications described herein, the biological or neural signals are used as control input to the application. The biological input can be used to control cursor movement on the screen of a graphical user interface (“GUI”), select options on the GUI, scroll up or down through a window, or other type of command or input used in computer applications. Within the realm of the computer application, haptic feedback can be sent to haptic devices 20 via haptic device interface 28 in response to an event within the realm of the computer application running on the computer.

Haptic feedback program 36 of the present disclosure can be implemented in hardware, software, firmware, or a combination thereof. When implemented in software or firmware, haptic feedback program 36 can be stored in memory and executed by a processing device, as explained above. When implemented in hardware, haptic feedback program 36 can be implemented, for example, using discrete logic circuitry, an application specific integrated circuit (“ASIC”), a programmable gate array (“PGA”), a field programmable gate array (“FPGA”), etc., or any combination thereof. The haptic feedback program 36 could also be implemented using analog circuitry. The programs or software code that include executable logical instructions, as described herein, can be embodied in any suitable computer-readable medium for execution by any suitable processing device. The computer-readable medium can include any physical medium that can store the programs or software code for a measurable length of time.

FIG. 4 is a flow chart illustrating a process for providing a haptic effect in response to detected neural activity, according to one embodiment. In block 38, a neural signal from a human is detected. It should be understood that the process of detecting, as described with respect to block 38, can alternatively include detecting neural signals from any mammal or vertebrate from which neural activity can be detected. Also, neural signals may be detected from the brain or from another part of the central nervous system. Signals may also be detected from other biological activity, such as eye movement, muscle movement or contraction, breathing rate, heart rate, etc. In response to the neural signal detected in block 38, the process of FIG. 4 can branch to a first branch including blocks 40 and 42, a second branch includes blocks 44, 46, 48, and 50, or both branches (as illustrated). When both branches are executed, a processing system, such as processor 22, for example, may perform the tasks in the second branch in parallel with the first branch.

In the first branch, block 40 includes generating a haptic response signal in response to the neural signal. In block 42, a haptic effect, related to the haptic response signal, is actuated on the human. In the second branch, block 44 includes processing the detected neural signal to control an external device. The external device can be a computer or other processor-based device. In some embodiments, the external device can be an electric wheelchair, a prosthetic device, or other type of robotic or movement-controllable device. The controlled device can also include any other suitable electronic device operating on input control signals. In block 46, an event is detected within the realm of the external device. Depending on the type of external device being controlled, the event can include any type of reaction, result, action, etc., which occurs to or with some portion of the external device. For example, in computer games, an event can be a controlled avatar interacting with other objects in the game. An avatar being struck by someone or something or experiencing a virtual sensation of some sort can be detected within the realm of the computer game.

In block 48, a haptic response signal is generated in response to the event detected in block 46. In this respect, the haptic response signal can be coordinated with the timing of the event. Also, the haptic response signal can be generated to correspond to the urgency, intensity, or other qualities of the event. Furthermore, different haptic response signals can be generated for different events to create a type of communication consistency with the user. In block 50, a haptic effect, based on the haptic response signal, is actuated upon the human being. Generally, the haptic response signal can be received by a haptic actuator capable of interpreting the signal and applying or simulating a sensation for the person corresponding to information in the signal.

It should be understood that the steps, processes, or operations described herein may represent any module or code sequence that can be implemented in software or firmware. In this regard, these modules and code sequences can include commands or instructions for executing specific logical steps, processes, or operations within physical components. It should further be understood that one or more of the steps, processes, and/or operations described herein may be executed substantially simultaneously or in a different order than explicitly described, as would be understood by one of ordinary skill in the art.

The embodiments described herein merely represent exemplary implementations and are not intended to necessarily limit the present disclosure to any specific examples. Instead, various modifications can be made to these embodiments as would be understood by one of ordinary skill in the art. Any such modifications are intended to be included within the spirit and scope of the present disclosure and protected by the following claims.

We claim:

1. An apparatus comprising:
a sensor configured to sense a neural signal from a human being;
a processing device in communication with the sensor, the processing device configured to receive a neural signal from the sensor and process the neural signal to generate a first signal;
a computer application configured to respond to the first signal and to generate a first haptic response signal as a result of an event within the computer application; and
a haptic device in communication with the computer application, the haptic device configured to receive the first haptic response signal and generate a first haptic effect on the human being.

2. The apparatus of claim 1, wherein the processing device is further configured to generate a second haptic response signal in response to receiving the neural signal, the haptic device further configured to receive the second haptic response signal and generate a second haptic effect on the human being, wherein the second haptic effect verifies to the human being when neural signals have been sensed.

3. The apparatus of claim 1, wherein each sensor and each haptic device is positioned adjacent to the skin of the human being.

4. The apparatus of claim 1, wherein the sensors detect neural activity from the central nervous system of the human being.

5. The apparatus of claim 1, wherein the sensors detect brain activity of the human being.

6. The apparatus of claim 1, further comprising a second sensor configured to detect one or more of a secondary set of inputs from the human being, the secondary set of inputs including eye movement, facial muscle movement, body muscle movement or contraction, breathing rate, and heart rate.

7. The apparatus of claim 1, wherein the haptic device applies a signal to the central nervous system of the human being to emulate the haptic effect.

8. The apparatus of claim 1, wherein the haptic device comprises one or more actuators, and the haptic effect is a vibrotactile effect.

9. A method comprising:
detecting a neural signal from a vertebrate; and
generating a haptic effect on the vertebrate corresponding to the neural signal.

10. The method of claim 9, wherein detecting the neural signal comprises detecting a plurality of neural signals from brain activity of the vertebrate.

11. The method of claim 9, wherein detecting the neural signal comprises emitting infrared radiation to detect blood flow.

12. The method of claim 9, further comprising:
detecting a secondary set of inputs from the vertebrate, the secondary set of inputs including at least one input selected from the group of inputs consisting of eye movement, body movement, muscle movement, and muscle contraction.

13. The method of claim 9, wherein actuating the haptic effect further comprises:
imposing a first haptic effect in response to the detection of the neural signal; and
imposing a second haptic effect in response to a detection of an event occurring in an external device controlled by the neural signal.

14. The method of claim 9, wherein generating the haptic effect comprises at least one of applying a vibration to the vertebrate with an actuator and applying an electrical output to one or more portions of the body of the vertebrate.

15. The method of claim 9, further comprising:
sensing a second neural signal from the vertebrate based on the vertebrate's response to the haptic effect; and
adjusting the haptic effect in response to the sensed second neural signal.

16. The method of claim 9, wherein the external device includes a computer and the detected event is associated with a location of a cursor within a graphical user interface of a computer application executed by the computer.

17. A system comprising:
means for sensing biological activity of a living organism;
means for processing the biological activity to generate a haptic response signal; and
means for rendering a haptic effect on the living organism, wherein the haptic effect is related to the haptic response signal generated by the processing means.

18. The system of claim 17, wherein the means for sensing biological activity includes means for sensing neural activity from the central nervous system of a human.

19. The system of claim 18, wherein the rendering means is configured to induce the haptic effect on the central nervous system of the human to mimic a sensation that occurs in reality.

20. The system of claim 17, further comprising:
means for interpreting the biological activity as a control signal to control an external electronic device.

21. The system of claim 20, wherein the means for rendering a haptic effect invokes a haptic effect based on an event occurring within the external electronic device.

22. A computer-readable medium adapted to store instructions that are executable by a processor, the stored instructions comprising logic configured to instruct the processor to:
detect a neural signal from a vertebrate; and
impose on the vertebrate a haptic effect corresponding to the neural signal.

23. The computer-readable medium of claim 22, wherein the stored instructions further comprise logic configured to instruct the processor, in response to the detected neural signal, to control a cursor or avatar in a computer program.

24. The computer-readable medium of claim 23, wherein the logic configured to instruct the processor to impose a haptic effect further comprises logic configured to instruct the processor to invoke a contraction of a muscle of the vertebrate to mimic the contraction of a virtual muscle of the avatar.

25. The computer-readable medium of claim 22, wherein the neural signal is related to brain activity.
